(19) United States
(12) Patent Application Publication
(10) Pub. No.: US 2025/0032460 A1
(43) Pub. Date: Jan. 30, 2025

(54) METHODS OF TREATING NEUROLOGICAL DISORDERS

(71) Applicant: MapLight Therapeutics, Inc., Redwood City, CA (US)

(72) Inventors: James LILLIE, Wellesley, MA (US); Michael WOOD, Gloucester, MA (US)

(21) Appl. No.: 18/712,916

(22) PCT Filed: Nov. 23, 2022
(86) PCT No.: PCT/US2022/080429

§ 371 Date: May 23, 2024

Related U.S. Application Data
(60) Provisional application No. 63/416,745, filed on Oct. 17, 2022.
(60) Provisional application No. 63/283,140, filed on Nov. 24, 2021.

(51) Int. Cl. A61K 31/4245; A61K 31/135; A61K 31/24; A61P 25/14

(52) U.S. Cl. CPC A61K 31/4245; A61K 31/135; A61K 31/24; A61P 25/14

(57) ABSTRACT

Methods of treating a neurological disorder (such as levodopa induced dyskinesia) by administering a combination of Compound I and fesoterodine.

### CROSS-REFERENCE TO RELATED APPLICATIONS

This application claims the benefit of U.S. Provisional Application No. 63/283,140, filed Nov. 24, 2021, and U.S. Provisional Application No. 63/416,745, filed Oct. 17, 2022, each of which is incorporated by reference for all purposes.

### BACKGROUND

Neurological disorders are now the leading source of disability in the world. Parkinson's disease (PD) is a progressive neurodegenerative disorder that affects movement. Parkinson's disease is the most common movement disorder and the fastest growing neurological disorder. In 2016, the global prevalence of Parkinson's disease was 6,062,893 with 3.2 million disability-adjusted life-years (DALYs) and 211,296 deaths. This is significantly higher than prevalence reported in 1990, when the global PD population was 2.5 million. In the United States in 2017, there were an estimated one million individuals with diagnosed Parkinson's disease. As the population ages, further substantial increases in PD diagnosis are projected. The projected PD prevalence in the year 2037 in the United States alone will be more than 1.6 million.

The motor symptoms arising from the loss of striatal dopamine (DA) in PD are routinely treated with levodopa (L-DOPA) treatment. Long-term levodopa therapy results in the development of abnormal involuntary movements called levodopa-induced dyskinesia (LID). Current treatment options for LID are limited and have many side effects. New methods of treating dyskinesias, including LID in Parkinson's patients, are needed.

The present disclosure provides methods of treating neurological disorders, such as dyskinesias (including LID).

### SUMMARY OF THE DISCLOSURE

In embodiments, the present disclosure provides methods of treating a neurological disorder in a patient in need thereof, the method comprising administering:
(a) a therapeutically effective amount of Compound 1, or a pharmaceutically acceptable salt thereof and
(b) a therapeutically effective amount of fesoterodine, or a pharmaceutically acceptable salt thereof.

In embodiments, the neurological disorder is a dyskinesia.

In embodiments, the dyskinesia is levodopa-induced dyskinesia.

In embodiments, the patient is diagnosed with Parkinson's disease.

In embodiments, the patient is treated for schizophrenia.

In embodiments, the neurological disorder is Alzheimer's disease psychosis, Parkinson's Disease Psychosis, Dementia Related Psychosis, Dementia with Lewy Bodies, Schizophrenia (acute and maintenance), brief psychotic disorder or Acute delirium.

In embodiments, about 5 mg to about 800 mg of Compound 1, or a pharmaceutically acceptable salt thereof is administered to the patient. In embodiments, about 20 mg to about 80 mg of Compound 1, or a pharmaceutically acceptable salt thereof is administered to the patient. In embodiments, about 60 mg of Compound 1, or a pharmaceutically acceptable salt thereof is administered to the patient.

In embodiments, the administration provides a therapeutically effective steady-state plasma concentration of Compound 1. In embodiments, the therapeutically effective steady-state plasma concentration of Compound 1 is about 100 ng/ml to 2500 ng/mL. In embodiments, the therapeutically effective steady-state plasma concentration of Compound 1 is about 600 ng/mL.

In embodiments, the Compound 1, or a pharmaceutically acceptable salt thereof is administered once per day. In embodiments, the Compound 1, or a pharmaceutically acceptable salt thereof is administered twice per day. In embodiments, the Compound 1, or a pharmaceutically acceptable salt thereof is administered three times per day.

In embodiments, about 1 mg to about 50 mg of fesoterodine, or a pharmaceutically acceptable salt thereof is administered to the patient. In embodiments, about 8 mg of fesoterodine, or a pharmaceutically acceptable salt thereof is administered. In embodiments, about 24 mg of fesoterodine, or a pharmaceutically acceptable salt thereof is administered.

In embodiments, the administration provides a therapeutically effective steady-state plasma concentration of desfesoterodine (i.e., an amount sufficient to reduce peripheral side effects associated with administration of Compound 1). In embodiments, the therapeutically effective steady-state plasma concentration of fesoterodine is about 5 ng/ml to about 30 ng/mL.

In embodiments, the fesoterodine, or a pharmaceutically acceptable salt thereof is administered once per day. In embodiments, the fesoterodine, or a pharmaceutically acceptable salt thereof is administered twice per day. In embodiments, the fesoterodine, or a pharmaceutically acceptable salt thereof is administered three times per day.

In embodiments, the Compound 1 and fesoterodine are administered in separate pharmaceutical compositions.

In embodiments, the Compound 1 and fesoterodine are orally administered.

In embodiments, the patient is administered the Compound 1 at least one hour after the patient is administered the fesoterodine.

In embodiments, the administration provides a Compound 1 to desfesoterodine plasma concentration ratio of about 10:1 to about 1000:1. In embodiments, the administration provides a Compound 1 to desfesoterodine plasma concentration ratio 100:1.

In embodiments, the dose and administration schedule of fesoterodine are selected to reduce the peripheral side effects of administering Compound 1 to the patient.

### BRIEF DESCRIPTION OF THE DRAWINGS

FIG. 1 shows the change from baseline salivation (mL) following administration of different doses of Compound 1 (1 mg, 3 mg, 10 mg, 20 mg, and 40 mg) as described in Example 7.

FIG. 2 shows the change from baseline salivation (mL) following administration of Compound 1 alone (40 mg) and following administration of Compound 1 (40 mg) in combination with fesoterodine fumarate (8 mg) as described in Example 7.

### DETAILED DESCRIPTION OF THE INVENTION

### Definitions

Throughout this disclosure, various patents, patent applications and publications (including non-patent publications) are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, the term “Compound 1” refers to 1-(3-methyl-[1,2,4]oxadiazol-5-yl)-(1R,5R)-3-aza-bicyclo[3.1.0]hexane having the following structural formula:

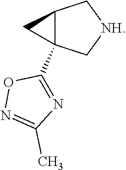

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “therapeutically effective amount” refers to that amount of a compound or pharmaceutical composition described herein that is sufficient to effect the intended application including, but not limited to, disease treatment, as illustrated below. For example, an effective amount of Compound 1 is that amount that is required to reduce at least one symptom of dyskinesia in a patient. An effective amount of fesoterodine is that amount that is required to reduce at least one side effect associated with administration of Compound 1, including, for example, salivation, lacrimation, urination, defecation, gastrointestinal distress and emesis, diarrhea, miosis, bronchorrhea, bronchospasms, laxation, and sweating. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the dosing regimen to be followed, timing of administration, the tissue to which it is administered, and the physical delivery system in which it is carried.

The term “therapeutic effect” as used herein refers to a desired or beneficial effect provided by the method and/or the composition. For example, the method for treating dyskinesia provides a therapeutic effect when the method reduces at least one symptom of dyskinesia in a patient.

The term “treating” as used herein with regard to a patient, refers to improving at least one symptom of the patient's disorder (for example, dyskinesia). Treating can be improving, or at least partially ameliorating a disorder.

### Methods of the Present Disclosure

Parkinson's disease (PD) results in primary motor symptoms, including tremors, rigidity, bradykinesia (slow movement) and postural instability (balance problems). The motor symptoms that arise from the loss of striatal dopamine (DA) in PD continues to be best relieved with levodopa (L-dopa) treatment. Levodopa, which is biosynthetically transformed to dopamine via aromatic L-amino acid decarboxylase (AADC), is commonly given to augment dopamine levels in PD patients.

Long-term levodopa therapy results in the development of abnormal involuntary movements called levodopa-induced dyskinesia (LID), which can develop as early as a few months after treatment and affects most patients after 5 to 10 years of L-DOPA therapy Levodopa-induced dyskinesia can comprise a variety of movement disorders including chorea, dystonia, ballism, myoclonus, and akathisia, and eventually become incapacitating with associated significant increase in treatment cost.

Treatment options for LID are inadequate. To date, the only approved pharmacotherapy, N-methyl-D-aspartate (NMDA)-type glutamate receptor antagonist amantadine, which exhibits variable efficacy and its use has been associated with tolerability issues.

The present disclosure provides methods of treating levodopa-induced dyskinesia (LID) by administering therapeutically effective amounts of Compound 1 (an M1/M4 receptor agonist). As described in the present Examples (see Example 4), the administration of Compound 1 provides a reduction in LID symptoms as determined using a standard LID disease model. Thus, Compound 1 is useful in treating dyskinesias (including LID). However, muscarinic receptor agonists (such as Compound 1) exhibit undesired peripheral cholinergic effects.

Fesoterodine is a non-selective antimuscarinic agent prodrug of 5-hydroxymethyl tolterodine (5-HMT) with affinity for M1, M2, M3, M4, and M5 muscarinic receptors that is used in the methods of the present disclosure to reduce the peripheral cholinergic effects of the administration of Compound 1.

In embodiments, the fesoterodine reduces the side effects associated with administration of Compound 1. In embodiments, the method of treating dyskinesia reduces side effects associated with administration of Compound 1, including, but not limited to, salivation, lacrimation, urination, defecation, gastrointestinal distress and emesis, diarrhea, miosis, bronchorrhea, bronchospasms, laxation, and sweating. In embodiments, the methods of present disclosure reduce peripheral side effects associated with administration of Compound 1. In embodiments, the methods of present disclosure reduce central side effects associated with administration of Compound 1, such as headache, confusion, and drowsiness.

The present disclosure provides methods of administering a muscarinic receptor agonist (e.g., Compound 1) in combination with a peripherally-acting anti-cholinergic agent (e.g., fesoterodine) to provide methods that effectively treat a dyskinesia with reduced peripheral cholinergic effects.

In embodiments, the present disclosure provides a method of treating a dyskinesia in a patient in need thereof, the method comprising administering:
(a) a therapeutically effective amount of Compound 1, or a pharmaceutically acceptable salt thereof and
(b) a therapeutically effective amount of fesoterodine, or a pharmaceutically acceptable salt thereof.

In embodiments, the dyskinesia is levodopa-induced dyskinesia.

In embodiments, the patient is diagnosed with Parkinson's disease.

In embodiments, the present disclosure provides a method of treating Alzheimer's disease psychosis, Parkinson's Disease Psychosis, Dementia Related Psychosis, Dementia with Lewy Bodies, Schizophrenia (acute and maintenance), brief psychotic disorder or Acute delirium in a patient in need thereof, the method comprising administering:
(a) a therapeutically effective amount of Compound 1, or a pharmaceutically acceptable salt thereof and
(b) a therapeutically effective amount of fesoterodine, or a pharmaceutically acceptable salt thereof.

In embodiments, the patient is treated for schizophrenia. In embodiments, the patient is treated for acute schizophrenia.

In embodiments, about 5 mg to about 300 mg of Compound 1, or a pharmaceutically acceptable salt thereof is administered to the patient. In embodiments, about 5 mg to about 800 mg of Compound 1, or a pharmaceutically acceptable salt thereof is administered to the patient, e.g., about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 22 mg, about 24 mg, about 26 mg, about 28 mg, about 30 mg, about 32 mg, about 34 mg, about 36 mg, about 38 mg, about 40 mg, about 42 mg, about 44 mg, about 46 mg, about 48 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg, including all values and ranges in between. In embodiments, about 20 mg to about 80mg of Compound 1, or a pharmaceutically acceptable salt thereof is administered to the patient. In embodiments, about 20 mg to about 60 mg of Compound 1, or a pharmaceutically acceptable salt thereof is administered to the patient.

In embodiments, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 22 mg, about 24 mg, about 26 mg, about 28 mg, about 30 mg, about 32 mg, about 34 mg, about 36 mg, about 38 mg, about 40 mg, about 42 mg, about 44 mg, about 46 mg, about 48 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, bout 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg of Compound 1, or a pharmaceutically acceptable salt thereof is administered to the patient. In embodiments about 40 mg of Compound 1, or a pharmaceutically acceptable salt thereof is administered to the patient. In embodiments about 60 mg of Compound 1, or a pharmaceutically acceptable salt thereof is administered to the patient. In embodiments about 80 mg of Compound 1, or a pharmaceutically acceptable salt thereof is administered to the patient.

In embodiments, about 0.05 mg/kg to about 8 mg/kg of Compound 1, or a pharmaceutically acceptable salt thereof is administered to the patient, e.g., about 0.05 mg/kg, about 0.10 mg/kg, about 0.15 mg/kg, about 0.20 mg/kg, about 0.25 mg/kg, about 0.30 mg/kg, about 0.35 mg/kg, about 0.40 mg/kg, about 0.45 mg/kg, about 0.50 mg/kg, about 0.55 mg/kg, about 0.60 mg/kg, about 0.65 mg/kg, about 0.70 mg/kg, about 0.75 mg/kg, about 0.80 mg/kg, about 0.85 mg/kg, about 0.90 mg/kg, about 0.95 mg/kg, about 1.00 mg/kg, about 1.20 mg/kg, about 1.40 mg/kg, about 1.60 mg/kg, about 1.80 mg/kg, about 2.00 mg/kg, about 2.20 mg/kg, about 2.40 mg/kg, about 2.60 mg/kg, about 2.80 mg/kg, about 3.00 mg/kg, about 3.20 mg/kg, about 3.40 mg/kg, about 3.60 mg/kg, about 3.80 mg/kg, about 4.00 mg/kg, about 4.20 mg/kg, about 4.40 mg/kg, about 4.60 mg/kg, about 4.80 mg/kg, about 5.00 mg/kg, about 5.20 mg/kg, about 5.40 mg/kg, about 5.60 mg/kg, about 5.80 mg/kg, about 6.00 mg/kg, about 6.20 mg/kg, about 6.40 mg/kg, about 6.60 mg/kg, about 6.80 mg/kg, about 7.00 mg/kg, about 7.20 mg/kg, about 7.40 mg/kg, about 7.60 mg/kg, about 7.80 mg/kg, or about 8.00 mg/kg, including all values and ranges in between.

In embodiments, the methods of the present disclosure provide a therapeutically effective blood plasma concentration of Compound 1 as measured by a patient's steady-state plasma concentration of Compound 1. In embodiments, the therapeutically effective steady-state plasma concentration of Compound 1 is about 200 ng/ml to about 1500 ng/ml. In embodiments, the therapeutically effective steady-state plasma concentration of Compound 1 is about 100 ng/ml to about 2500 ng/ml, e.g., about 100 ng/ml, about 110 ng/ml, about 120 ng/ml, about 130 ng/mL, about 140 ng/ml, about 150 ng/mL, about 160 ng/ml, about 170 ng/ml, about 280 ng/mL, about 190 ng/ml, about 200 ng/mL, about 210 ng/ml, about 220 ng/ml, about 230 ng/mL, about 240 ng/mL, about 250 ng/ml, about 260 ng/ml, about 270 ng/mL, about 280 ng/mL, about 290 ng/mL, about 300 ng/mL, about 310 ng/ml, about 320 ng/ml, about 330 ng/mL, about 340 ng/mL, about 350 ng/ml, about 360 ng/ml, about 370 ng/mL, about 380 ng/mL, about 390 ng/mL, about 400 ng/ml, about 410 ng/ml, about 420 ng/mL, about 430 ng/mL, about 440 ng/mL, about 450 ng/ml, about 460 ng/ml, about 470 ng/mL, about 480 ng/ml, about 490 ng/mL, about 500 ng/ml, about 510 ng/ml, about 520 ng/mL, about 530 ng/mL, about 540 ng/mL, about 550 ng/ml, about 560 ng/ml, about 570 ng/mL, about 580 ng/ml, about 590 ng/mL, about 600 ng/ml, about 610 ng/ml, about 620 ng/ml, about 630 ng/mL, about 640 ng/ml, about 650 ng/ml, about 660 ng/ml, about 670 ng/ml, about 680 ng/mL, about 690 ng/mL, about 700 ng/ml, about 710 ng/ml, about 720 ng/ml, about 730 ng/mL, about 740 ng/mL, about 750 ng/mL, about 760 ng/ml, about 770 ng/mL, about 780 ng/mL, about 790 ng/ml, about 800 ng/ml, about 810 ng/ml, about 820 ng/ml, about 830 ng/mL, about 840 ng/mL, about 850 ng/ml, about 860 ng/ml, about 870 ng/mL, about 880 ng/ml, about 890 ng/mL, about 900 ng/ml, about 910 ng/ml, about 920 ng/mL, about 930 ng/mL, about 940 ng/ml, about 950 ng/ml, about 960 ng/ml, about 970 ng/ml, about 980 ng/mL, about 990 ng/mL, about 1000 ng/ml, about 1050 ng/ml, about 1100 ng/ml, about 1150 ng/ml, about 1200 ng/mL, about 1250 ng/ml, about 1300 ng/ml, about 1350 ng/mL, about 1400 ng/mL, about 1450 ng/mL, about 1500 ng/ml, about 1550 ng/ml, about 1600 ng/ml, about 1650 ng/ml, about 1700 ng/ml, about 1750 ng/ml, about 1800 ng/mL, about 1850 ng/ml, about 1900 ng/ml, about 1950 ng/ml, or about 2000 ng/mL, about 2050 ng/mL, about 2100 ng/mL, about 2150 ng/ml, about 2200 ng/ml, about 2250 ng/ml, about 2300 ng/ml, about 2350 ng/mL, about 2400 ng/mL, about 2450 ng/mL, or about 2500 ng/mL, including all values and ranges in between. In embodiments, the therapeutically effective steady-state plasma concentration of Compound 1 is about 500 ng/ml to 1000 ng/mL. In embodiments, the therapeutically effective steady-state plasma concentration of Compound 1 is about 600 ng/mL.

In embodiments, the methods of the present disclosure provide a therapeutically effective blood plasma concentration of Compound 1 as measured by a patient's average maximum blood concentration (Cₘₐₓ) of Compound 1. In embodiments, the methods of the present disclosure provide an average maximum blood plasma concentration (Cₘₐₓ) of Compound 1 of about 400 ng/ml to about 2500 ng/mL, e.g., about 400 ng/ml, about 410 ng/ml, about 420 ng/mL, about 430 ng/mL, about 440 ng/mL, about 450 ng/mL, about 460 ng/mL, about 470 ng/mL, about 480 ng/ml, about 490 ng/ml, about 500 ng/mL, about 510 ng/mL, about 520 ng/mL, about 530 ng/mL, about 540 ng/ml, about 550 ng/mL, about 560 ng/ml, about 570 ng/mL, about 580 ng/ml, about 590 ng/ml, about 600 ng/mL, about 610 ng/ml, about 620 ng/mL, about 630 ng/mL, about 640 ng/ml, about 650 ng/mL, about 660 ng/mL, about 670 ng/ml, about 680 ng/ml, about 690 ng/ml, about 700 ng/ml, about 710 ng/ml, about 720 ng/mL, about 730 ng/mL, about 740 ng/ml, about 750 ng/ml, about 760 ng/mL, about 770 ng/mL, about 780 ng/ml, about 790 ng/mL, about 800 ng/ml, about 810 ng/ml, about 820 ng/ml, about 830 ng/mL, about 840 ng/mL, about 850 ng/ml, about 860 ng/ml, about 870 ng/mL, about 880 ng/ml, about 890 ng/ml, about 900 ng/ml, about 910 ng/ml, about 920 ng/mL, about 930 ng/ml, about 940 ng/ml, about 950 ng/ml, about 960 ng/mL, about 970 ng/mL, about 980 ng/mL, about 990 ng/ml, about 1000 ng/mL, about 1050 ng/mL, about 1100 ng/mL, about 1150 ng/mL, about 1200 ng/ml, about 1250 ng/ml, about 1300 ng/mL, about 1350 ng/mL, about 1400 ng/mL, about 1450 ng/ml, about 1500 ng/ml, about 1550 ng/mL, about 1600 ng/ml, about 1650 ng/ml, about 1700 ng/ml, about 1750 ng/mL, about 1800 ng/mL, about 1850 ng/mL, about 1900 ng/ml, about 1950 ng/ml, or about 2000 ng/mL, about 2050 ng/ml, about 2100 ng/ml, about 2150 ng/ml, about 2200 ng/ml, about 2250 ng/mL, about 2300 ng/ml, about 2350 ng/mL, about 2400 ng/mL, about 2450 ng/mL, or about 2500 ng/mL, including all values and ranges in between. In embodiments, the methods of the present disclosure provide a Cₘₐₓ of Compound 1 of about 2500 ng/ml. In embodiments, the methods of the present disclosure provide a Cₘₐₓ of Compound 1 of about 1000 ng/mL. In embodiments, the methods of the present disclosure provide a Cₘₐₓ of Compound 1 of about 400 ng/mL.

In embodiments, the methods of the present disclosure provide a therapeutically effective blood plasma concentration of Compound 1 as measured by a patient's average minimum blood plasma concentration (Cₘᵢₙ) of Compound 1. In embodiments, the methods of the present disclosure provide an average minimum blood plasma concentration (Cₘᵢₙ) of Compound 1 of about 200 ng/ml to about 1000 ng/ml, e.g., about 200 ng/ml, about 210 ng/mL, about 220 ng/mL, about 230 ng/ml, about 240 ng/ml, about 250 ng/ml, about 260 ng/ml, about 270 ng/ml, about 280 ng/mL, about 290 ng/mL, about 300 ng/ml, about 310 ng/ml, about 320 ng/mL, about 330 ng/mL, about 340 ng/mL, about 350 ng/ml, about 360 ng/mL, about 370 ng/mL, about 380 ng/mL, about 390 ng/ml, about 400 ng/mL, about 410 ng/mL, about 420 ng/mL, about 430 ng/mL, about 440 ng/ml, about 450 ng/mL, about 460 ng/mL, about 470 ng/mL, about 480 ng/mL, about 490 ng/ml, about 500 ng/ml, about 510 ng/ml, about 520 ng/ml, about 530 ng/mL, about 540 ng/ml, about 550 ng/ml, about 560 ng/ml, about 570 ng/mL, about 580 ng/ml, about 590 ng/mL, about 600 ng/ml, about 610 ng/ml, about 620 ng/mL, about 630 ng/mL, about 640 ng/ml, about 650 ng/ml, about 660 ng/mL, about 670 ng/ml, about 680 ng/mL, about 690 ng/ml, about 700 ng/ml, about 710 ng/mL, about 720 ng/mL, about 730 ng/mL, about 740 ng/ml, about 750 ng/ml, about 760 ng/ml, about 770 ng/mL, about 780 ng/ml, about 790 ng/ml, about 800 ng/ml, about 810 ng/mL, about 820 ng/ml, about 830 ng/mL, about 840 ng/ml, about 850 ng/ml, about 860 ng/ml, about 870 ng/ml, about 880 ng/mL, about 890 ng/mL, about 900 ng/ml, about 910 ng/mL, about 920 ng/mL, about 930 ng/ml, about 940 ng/ml, about 950 ng/mL, about 960 ng/ml, about 970 ng/ml, about 980 ng/mL, about 990 ng/mL, or about 1000 ng/mL, including all values and ranges in between.

In embodiments, the dose and administration schedule of fesoterodine is selected to reduce the peripheral side effects of administering Compound 1 to the patient treated for a neurological disorder. In embodiments, the dose and administration schedule of fesoterodine is selected to reduce the peripheral side effects of administering Compound 1 to the patient treated for a dyskinesia (e.g., LID).

In embodiments, about 1 mg to about 20 mg of fesoterodine, or a pharmaceutically acceptable salt thereof is administered to the patient. In embodiments, about 1 mg to about 50 mg of fesoterodine, or a pharmaceutically acceptable salt thereof is administered to the patient, e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, 41 mg, about 42 mg, about 43 mg, about 44 mg, about 45 mg, about 46 mg, about 47 mg, about 48 mg, about 49 mg, or about 50 mg including all values and ranges in between. In embodiments, about 3mg of fesoterodine, or a pharmaceutically acceptable salt thereof is administered. In embodiments, about 4 mg of fesoterodine, or a pharmaceutically acceptable salt thereof is administered. In embodiments, about 6 mg of fesoterodine, or a pharmaceutically acceptable salt thereof is administered. In embodiments, about 8 mg of fesoterodine, or a pharmaceutically acceptable salt thereof is administered. In embodiments, about 24 mg of fesoterodine, or a pharmaceutically acceptable salt thereof is administered.

In embodiments, about 0.01 mg/kg to about 0.602 mg/kg of fesoterodine, or a pharmaceutically acceptable salt thereof is administered to the patient, e.g., about 0.010 mg/kg, about 0.011 mg/kg, about 0.012 mg/kg, about 0.013 mg/kg, about 0.015 mg/kg, about 0.016 mg/kg, about 0.018 mg/kg, about 0.019 mg/kg, about 0.021 mg/kg, about 0.024 mg/kg, about 0.026 mg/kg, about 0.029 mg/kg, about 0.031 mg/kg, about 0.035 mg/kg, about 0.038 mg/kg, about 0.042 mg/kg, about 0.046 mg/kg, about 0.051 mg/kg, about 0.056 mg/kg, about 0.061 mg/kg, about 0.067 mg/kg, about 0.074 mg/kg, about 0.081 mg/kg, about 0.090 mg/kg, about 0.098 mg/kg, about 0.108 mg/kg, about 0.119 mg/kg, about 0.131 mg/kg, about 0.144 mg/kg, about 0.159 mg/kg, about 0.174 mg/kg, about 0.192 mg/kg, about 0.211 mg/kg, about 0.232 mg/kg, about 0.255 mg/kg, about 0.281 mg/kg, about 0.309 mg/kg, about 0.340 mg/kg, about 0.374 mg/kg, about 0.411 mg/kg, about 0.453 mg/kg, about 0.498 mg/kg, about 0.548 mg/kg, about 0.602 mg/kg, including all values and ranges in between.

In embodiments, the Compound 1, or a pharmaceutically acceptable salt thereof and fesoterodine, or a pharmaceutically acceptable salt thereof are administered to the patient in a weight ratio of Compound 1 to fesoterodine of about 2:1 to 50:1, e.g., about 2:1, about 3:1,about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, to about 50:1, including all values and ranges in between. In embodiments, the Compound 1, or a pharmaceutically acceptable salt thereof, and fesoterodine, or a pharmaceutically acceptable salt thereof, are administered to the patient in a weight ratio of Compound 1 to fesoterodine of about 2:1 to 30:1. In embodiments, the Compound 1, or a pharmaceutically acceptable salt thereof, and fesoterodine, or a pharmaceutically acceptable salt thereof, are administered to the patient in a ratio of Compound 1 to fesoterodine of about 5:1 to 15:1.

In embodiments, the fesoterodine is administered in a sustained release composition.

In embodiments, the administration provides a therapeutically effective steady-state plasma concentration of desfesoterodine (i.e., an amount sufficient to reduce peripheral side effects or CNS side effects associated with administration of Compound 1).

In embodiments, the methods of the present disclosure provide a therapeutically effective blood plasma concentration of desfesoterodine (i.e., a metabolite of fesoterodine) as measured by a patient's steady-state plasma concentration of desfesoterodine. In embodiments, the methods of the present disclosure provide a therapeutically effective steady-state plasma concentration of desfesoterodine of about 50 ng/ml to about 20 ng/mL. In embodiments, the methods of the present disclosure provide a therapeutically effective steady-state plasma concentration of desfesoterodine of about 0.1 ng/mL to about 30 ng/mL. In embodiments, the methods of the present disclosure provide a therapeutically effective steady-state plasma concentration of desfesoterodine of about 50 pg/mL to about 30 ng/ml, e.g. about 50 pg/mL, about 60 pg/mL, 70 pg/mL, about 80 pg/mL, about 90 pg/mL, about 0.1 ng/ml, about 0.2 ng/ml, about 0.3 ng/ml, about 0.4 ng/ml, about 0.5 ng/ml, about 0.6 ng/ml, about 0.7 ng/ml, about 0.8 ng/ml, about 0.9 ng/ml, about 1.0 ng/mL, about 1.5 ng/ml, about 2.0 ng/ml, about 2.5 ng/ml, about 3.0 ng/mL, about 3.5 ng/ml, about 4.0 ng/ml, about 4.5 ng/ml, about 5.0 ng/ml, about 5.5 ng/ml, about 6.0 ng/mL, about 6.5 ng/ml, about 7.0 ng/ml, about 7.5 ng/ml, about 8.0 ng/ml, about 8.5 ng/ml, about 9.0 ng/ml, about 9.5 ng/mL, about 10.0 ng/ml, about 10.5 ng/ml, about 11.0 ng/ml, about 11.5 ng/ml, about 12.0 ng/ml, about 12.5 ng/mL, about 13.0 ng/mL, about 13.5 ng/ml, about 14.0 ng/ml, about 14.5 ng/ml, about 15.0 ng/mL, about 15.5 ng/ml, about 16.0 ng/ml, about 16.5 ng/ml, about 17.0 ng/mL, about 17.5 ng/ml, about 18.0 ng/ml, about 18.5 ng/ml, about 19.0 ng/ml, about 19.5 ng/mL, about 20.0 ng/ml, about 20.5 ng/mL, about 21.0 ng/ml, about 21.5 ng/ml, about 22.0 ng/ml, about 22.5 ng/mL, about 23.0 ng/ml, about 23.5 ng/ml, about 24.0 ng/ml, about 24.5 ng/ml, about 25.0 ng/mL, about 25.5 ng/ml, about 26.0 ng/ml, about 26.5 ng/mL, about 27.0 ng/ml, about 27.5 ng/ml, about 28.0 ng/ml, about 28.5 ng/ml, about 29.0 ng/mL, about 29.5 ng/mL, to about 30.0 ng/mL, including all values and ranges in between. In embodiments, the administration provides a therapeutically effective steady-state plasma concentration of desfesoterodine of about 5 ng/ml to about 10 ng/ml.

In embodiments, the Compound 1, or a pharmaceutically acceptable salt thereof is administered once per day. In embodiments, the Compound 1, or a pharmaceutically acceptable salt thereof is administered twice per day. In embodiments, the Compound 1, or a pharmaceutically acceptable salt thereof is administered three times per day.

In embodiments, the fesoterodine, or a pharmaceutically acceptable salt thereof is administered once per day. In embodiments, the fesoterodine, or a pharmaceutically acceptable salt thereof is administered twice per day. In embodiments, the fesoterodine, or a pharmaceutically acceptable salt thereof is administered three times per day.

In embodiments, the Compound 1 and fesoterodine are orally administered. In embodiments, the Compound 1 is orally administered. In embodiments, the fesoterodine is orally administered.

In embodiments, the Compound 1 and fesoterodine are intravenously administered. In embodiments, the Compound 1 is intravenously administered. In embodiments, the fesoterodine is intravenously administered.

In embodiments, the patent is administered fesoterodine and Compound 1 according to a dosing schedule that minimizes the side effects associated with Compound 1. In embodiments, the patient is administered the Compound 1 about 1 hour to about 10 hours after the patient is administered the fesoterodine. In embodiments, the patient is administered the Compound 1 about 5 minutes to about 10 hours after the patient is administered the fesoterodine, e.g., about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, or about 10 hours, including all values and ranges in between. In embodiments, the patient is administered the Compound 1 at least one hour after the patient is administered the fesoterodine. In embodiments, the patient is administered Compound 1 about four hours after the patient is administered the fesoterodine.

In embodiments, the methods of the present disclosure provide a Compound 1 to desfesoterodine plasma concentration ratio of about 10:1 to about 1000:1. In embodiments, the methods of the present disclosure provide a Compound 1 to desfesoterodine plasma concentration ratio of about 1:1 to about 1000:1, e.g., about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, about 50:1, about 51:1, about 52:1, about 53:1, about 54:1, about 55:1, about 56:1, about 57:1, about 58:1, about 59:1, about 60:1, about 61:1, about 62:1, about 63:1, about 64:1, about 65:1, about 66:1, about 67:1, about 68:1, about 69:1, about 70:1, about 71:1, about 72:1, about 73:1, about 74:1, about 75:1, about 76:1, about 77:1, about 78:1, about 79:1, about 80:1, about 81:1, about 82:1, about 83:1, about 84:1, about 85:1, about 86:1, about 87:1, about 88:1, about 89:1, about 90:1, about 91:1, about 92:1, about 93:1, about 94:1, about 95:1, about 96:1, about 97:1, about 98:1, about 99:1, about 100:1, about 110:1, about 120:1, about 130:1, about 140:1, about 150:1, about 160:1, about 170:1, about 180:1, about 190:1, about 200:1, about 210:1, about 220:1, about 230:1, about 240:1, about 250:1, about 260:1, about 270:1, about 280:1, about 290:1, about 300:1, about 310:1, about 320:1, about 330:1, about 340:1, about 350:1, about 360:1, about 370:1, about 380:1, about 390:1, about 400:1, about 410:1, about 420:1, about 430:1, about 440:1, about 450:1, about 460:1, about 470:1, about 480:1, about 490:1, about 500:1, about 550:1, about 600:1, about 650:1, about 700:1, about 750:1, about 800:1, about 850:1, about 900:1, about 950:1, about 1000:1, including all values and ranges in between. In embodiments, the administration provides a Compound 1 to desfesoterodine plasma concentration ratio of about 100:1. In embodiments, the administration provides a Compound 1 to desfesoterodine plasma concentration ratio of about 470:1.

In embodiments, the Compound 1 and fesoterodine are administered in the same pharmaceutical composition. In embodiments, the Compound 1 and fesoterodine are administered as separate pharmaceutical compositions.

In embodiments, the fesoterodine, or a pharmaceutically acceptable salt thereof comprises fesoterodine fumarate.

### Pharmaceutical Formulations

The methods of the present disclosure can employ various formulations for administration to patients, e.g., humans, in unit dosage forms, such as tablets, capsules, films, orally disintegrating tablets, pills, powders, granules, sterile parenteral solutions or suspensions (e.g., intramuscular (IM), subcutaneous (SC) and intravenous (IV)), transdermal patches, and oral solutions or suspensions, and oil-water emulsions.

Oral pharmaceutical dosage forms can be either solid or liquid. The solid dosage forms can be tablets, capsules, granules, films (e.g., buccal films) and bulk powders. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art. In some embodiments, the present oral dosage forms may include orally disintegrating tablets. In some embodiments, the oral dosage forms is one or more oral extended release tablets.

In embodiments, pharmaceutical formulations may comprise one or more pharmaceutically acceptable excipients or adjuvants. The pharmaceutically acceptable excipients and adjuvants are added to the composition or formulation for a variety of purposes. In embodiments, the pharmaceutical formulations may comprise a pharmaceutically acceptable carrier. In embodiments, a pharmaceutically acceptable carrier includes a pharmaceutically acceptable excipient, binder, and/or diluent. In embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone.

In embodiments, the disclosure provides a pharmaceutical composition comprising Compound 1 and one or more pharmaceutically acceptable carriers or excipients.

In embodiments, the disclosure provides a pharmaceutical composition comprising fesoterodine and one or more pharmaceutically acceptable carriers or excipients.

In embodiments, the disclosure provides a pharmaceutical composition comprising Compound 1, fesoterodine and one or more pharmaceutically acceptable carriers or excipients.

### NUMBERED EMBODIMENTS

1. A method of treating a dyskinesia in a patient in need thereof, the method comprising administering:
   (a) a therapeutically effective amount of Compound 1, or a pharmaceutically acceptable salt thereof and
   (b) a therapeutically effective amount of fesoterodine, or a pharmaceutically acceptable salt thereof.
2. The method of embodiment 1, wherein the dyskinesia is levodopa-induced dyskinesia.
3. The method of any one of embodiments 1-2, wherein the patient is diagnosed with Parkinson's disease.
4. A method of treating Alzheimer's disease psychosis, Parkinson's Disease Psychosis, Dementia Related Psychosis, Dementia with Lewy Bodies, Schizophrenia (acute and maintenance), brief psychotic disorder or Acute delirium in a patient in need thereof, the method comprising administering:
   (a) a therapeutically effective amount of Compound 1, or a pharmaceutically acceptable salt thereof and
   (b) a therapeutically effective amount of fesoterodine, or a pharmaceutically acceptable salt thereof.
5. The method of embodiment 4, wherein the patient is treated for schizophrenia.
6. The method of any one of embodiments 4-5, wherein the patient is treated for Alzheimer's disease psychosis, Parkinson's Disease Psychosis, Dementia Related Psychosis, or Dementia with Lewy Bodies.
7. The method of any one of embodiments 1-6, wherein about 5 mg to about 800 mg of Compound 1, or a pharmaceutically acceptable salt thereof is administered to the patient.
7a. The method of any one of embodiments 1-6, wherein about 5 mg to about 300 mg of Compound 1, or a pharmaceutically acceptable salt thereof is administered to the patient.
8. The method of embodiment 7, wherein about 20 mg to about 80 mg of Compound 1, or a pharmaceutically acceptable salt thereof is administered to the patient.
9. The method of embodiment 7, wherein about 60 mg of Compound 1, or a pharmaceutically acceptable salt thereof is administered to the patient.
10. The method of any one of embodiments 1-9, wherein the administration provides a therapeutically effective steady-state plasma concentration of Compound 1.
11. The method of embodiment 10, wherein the therapeutically effective steady-state plasma concentration of Compound 1 is about 100 ng/ml to about 2500 ng/mL.
11a. The method of embodiment 10, wherein the therapeutically effective steady-state plasma concentration of Compound 1 is about 500 ng/ml to about 1000 ng/ml.
12. The method of embodiment 10, wherein the therapeutically effective steady-state plasma concentration of Compound 1 is about 600 ng/mL.
13. The method of any one of embodiments 1-12, wherein about 1 mg to about 50 mg of fesoterodine, or a pharmaceutically acceptable salt thereof is administered to the patient.
13a. The method of any one of embodiments 1-12, wherein about 1 mg to about 20 mg of fesoterodine, or a pharmaceutically acceptable salt thereof is administered to the patient.
13b. The method of any one of embodiments 1-12, wherein about 4 mg of fesoterodine, or a pharmaceutically acceptable salt thereof is administered to the patient.
14. The method of embodiment 13, wherein about 8 mg of fesoterodine, or a pharmaceutically acceptable salt thereof is administered.
15. The method of embodiment 13, wherein about 24 mg of fesoterodine, or a pharmaceutically acceptable salt thereof is administered.
16. The method of any one of embodiments 1-15, wherein the administration provides a therapeutically effective steady-state plasma concentration of desfesoterodine (i.e., an amount sufficient to reduce peripheral side effects associated with administration of Compound 1).
17. The method of embodiment 16, wherein the therapeutically effective steady-state plasma concentration of about 5 ng/ml to about 30 ng/mL.
17a. The method of embodiment 16, wherein the therapeutically effective steady-state plasma concentration of about 5 ng/mL to about 10 ng/ml.
18. The method of any one of embodiments 1-17, wherein the Compound 1, or a pharmaceutically acceptable salt thereof is administered once per day.
19. The method of any one of embodiments 1-17, wherein the Compound 1, or a pharmaceutically acceptable salt thereof is administered twice per day.
20. The method of any one of embodiments 1-17, wherein the Compound 1, or a pharmaceutically acceptable salt thereof is administered three times per day.
21. The method of any one of embodiments 1-20, wherein the fesoterodine, or a pharmaceutically acceptable salt thereof is administered once per day.
22. The method of any one of embodiments 1-20, wherein the fesoterodine, or a pharmaceutically acceptable salt thereof is administered twice per day.
23. The method of any one of embodiments 1-20, wherein the fesoterodine, or a pharmaceutically acceptable salt thereof is administered three times per day.
24. The method of any one of embodiments 1-23, wherein the Compound 1 and fesoterodine are administered in separate pharmaceutical compositions.
25. The method of any one of embodiments 1-24, wherein the Compound 1 and fesoterodine are orally administered.
26. The method of any one of embodiments 1-25, wherein the fesoterodine, or a pharmaceutically acceptable salt thereof is administered in an extended release composition.
27. The method of any one of embodiments 1-26, wherein the patient is administered the Compound 1 at least one hour after the patient is administered the fesoterodine.
28. The method of embodiment 27, wherein the patient is administered the Compound 1 about four hours after the patient is administered the fesoterodine.
29. The method of any one of embodiments 1-28, wherein the administration provides a Compound 1 to desfesoterodine plasma concentration ratio of about 10:1 to about 1000:1.
30. The method of any one of embodiments 1-28, wherein the administration provides a Compound 1 to desfesoterodine plasma concentration ratio of about 100:1.
31. The method of any one of embodiments 1-26 or 29-30, wherein the Compound 1 and fesoterodine are administered in the same pharmaceutical composition.
32. The method of any one of embodiments 1-31, wherein the Compound 1 comprises the hydrochloride salt of formula:

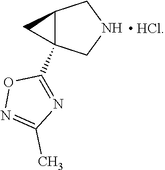

33. The method of any one of embodiments 1-32, wherein the fesoterodine, or a pharmaceutically acceptable salt thereof comprises fesoterodine fumarate.
34. The method of any one of embodiments 1-33, wherein the dose and administration schedule of fesoterodine are selected to reduce the peripheral side effects of administering Compound 1 to the patient.

### EXAMPLES

The present invention is further illustrated by reference to the following Examples. However, it is noted that these Examples are illustrative and are not to be construed as restricting the scope of the invention in any way.

### Example 1: Compound 1 Effect on Amphetamine-Induced Hyperlocomotion

The hyperdopaminergic state underlying LID was modeled in naïve male wild-type c57/B16 mice by administration of d-amphetamine, a drug which rapidly elevates synaptic dopamine levels.

A single dose of 3 mg/kg of intraperitoneal (IP) d-amphetamine was administered to 32 male wild-type c57/B16 mice at approximately 8-weeks of age. Subsequently, the animals were subdivided into 3 groups and co-administered either a single dose of intraperitoneal vehicle only, Compound 1 at 0.3 mg/kg, or Compound 1 at 1 mg/kg. The locomotor behavior of the animals in an open field chamber (44 cm×44 cm×20 cm dimension) was monitored using a video tracking system (Noldus EthoVision v15). Over 30 minutes, the average (mean) centimeters traveled (+standard deviation) identified were:
7500 cm (±314.9) in vehicle cohort
5141 cm (±805.6) in the Compound 1 0.3 mg/kg cohort (p<0.5 one-way ANOVA followed by Tukey's multiple comparison)
2008 cm (±406.2) in the Compound 1 1.0 mg/kg cohort (p <0.001)

The study demonstrated that Compound 1 reverses hyperdopaminergic-mediated motor behavior in a dose-dependent manner. These data suggest that Compound 1 may have utility in treating psychosis disorders, such as schizophrenia, Alzheimer's disease and Parkinson's disease as well levodopa-induced dyskinesia.

### Example 2: Compound 1 and Fesoterodine Dose-Response Effect on Salivation

An in vivo primary pharmacology study evaluating the fesoterodine fumarate dose-response antagonistic effect on Compound 1 induced salivation in anaesthetized male Sprague Dawley rats was conducted. Salivation was induced by intravenous administration of Compound 1 at 1 mg/kg. Intravenous fesoterodine doses were administered at 0.0078 mg/mL, 0.0156 mg/mL, 0.0312 mg/mL, 0.0625 mg/mL, 0.125 mg/mL, 0.25 mg/mL, and 0.5 mg/mL. Fesoterodine was dosed 10 minutes prior to dosage of Compound 1. All animals were normal with no observed abnormality detected in clinical signs or behavior. Salivation was measured by using oral dry swab in anesthetized rats with the calculated difference between the dry swab weight and the wet swab weight indicating the weight of saliva produced. Samples were collected post-dosing for one hour at 10-minute intervals.

Compound 1 demonstrated a significant effect in increase in salivation compared to 0.9% saline vehicle control. Fesoterodine effect with Compound 1 demonstrated a significant decrease of salivation in a consistent dose-responsive manner, as summarized in Table 1.

TABLE 1
Compound 1 and Fesoterodine Dose-Response Effect on Salivation

| | Salivation (mg) | | | | | | |
|---|---|---|---|---|---|---|---|
| | (mean value) | | | | | | |
| | 10 | 20 | 30 | 40 | 50 | 60 | |
| Dosing | min | min | min | min | min | min | Total |
| Control vehicle 0.9% | 3.68 | 1.5 | 2.17 | 0.71 | 0.57 | 0.87 | 9.49 |
| saline | | | | | | | |
| Compound 1 (1.5 mg/kg) | 414.44 | 126.95 | 59.21 | 61.22 | 24.79 | 22.15 | 708.76 |
| Fesoterodine 0.0078 | 347.49 | 90.03 | 26.31 | 10.29 | 9.96 | 7.74 | 491.83 |
| mg/mL | | | | | | | |
| Compound 1 (1.5 mg/kg) | | | | | | | |
| Fesoterodine 0.0156 | 163.43 | 69.8 | 30.58 | 16.26 | 11.66 | 5.71 | 297.45 |
| mg/mL | | | | | | | |
| Compound 1 (1.5 mg/kg) | | | | | | | |
| Fesoterodine 0.0312 | 95.37 | 61.86 | 29.68 | 8.31 | 8.78 | 6.64 | 210.64 |
| mg/mL | | | | | | | |
| Compound 1 (1.5 mg/kg) | | | | | | | |
| Fesoterodine 0.0625 | 66.8 | 27.58 | 12.89 | 9.18 | 6.56 | 2.45 | 125.47 |
| mg/mL | | | | | | | |
| Compound 1 (1.5 mg/kg) | | | | | | | |
| Fesoterodine 0.125 mg/mL | 45.22 | 21.91 | 7.85 | 5.06 | 4.73 | 3.5 | 88.27 |
| Compound 1 (1.5 mg/kg) | | | | | | | |
| Fesoterodine 0.25 mg/mL | 7 | 3.13 | 3.17 | 2.15 | 2.49 | 1.37 | 19.31 |
| Compound 1 (1.5 mg/kg) | | | | | | | |
| Fesoterodine 0.5 mg/mL | 7.23 | 2.83 | 1.21 | 4.11 | 2.5 | 1.8 | 19.69 |
| Compound 1 (1.5 mg/kg) | | | | | | | |

Abbreviations:
kg = kilogram,
mg = milligram,
min = minutes,
mL = milliliter

At the end of the study period, plasma samples were collected to measure exposure of Compound 1, fesoterodine fumarate, and fesoterodine active metabolite 5-hydroxymethyl tolterodine (5-HMT), also referred to as desfesoterodine, is summarized in Table 2.

TABLE 2
Post-Procedure Mean Plasma Exposure Compound 1, Fesoterodine,
Metabolite Desfesoterodine (5-HMT)

| | Plasma Concentration | | |
|---|---|---|---|
| | 1-Hour Post-procedure | | |
| | (mean value) | | |
| | | | Desfesoterodine |
| | Compound 1 | Fesoterodine | (5-HMT) |
| Dose | (ng/mL) | (ng/mL) | (ng/mL) |
| Compound 1 1.5 mg/kg | 608.00 | NA | NA |
| Fesoterodine 0.0078 mg/mL | 487.00 | No peak | BLQ |
| Compound 1 (1.5 mg/kg) | | | |
| Fesoterodine 0.0156 mg/mL | 686.52 | No peak | BLQ |
| Compound 1 (1.5 mg/kg) | | | |
| Fesoterodine 0.0312 mg/mL | 668.91 | No peak | BLQ |
| Compound 1 (1.5 mg/kg) | | | |
| Fesoterodine 0.0625 mg/mL | 729.52 | No peak | BLQ |
| Compound 1 (1.5 mg/kg) | | | |
| Fesoterodine 0.125 mg/mL | 773.95 | No peak | BLQ |
| Compound 1 (1.5 mg/kg) | | | |
| Fesoterodine 0.25mg/mL | 539.37 | No peak | 5.15 |
| Compound 1 (1.5 mg/kg) | | | |
| Fesoterodine 0.5 mg/mL | 757.95 | No peak | 6.19 |
| Compound 1 (1.5 mg/kg) | | | |

Abbreviations: 5-HMT = 5-hydroxymethyl tolterodine, BLQ = below limit of quantification, kg = kilogram, mg = milligram, mL = milliliter

Based upon the salivation and exposure data, a Compound 1:5-HMT plasma concentration ratio of 100:1 mitigates the Compound 1-induced peripheral cholinergic activity of salivation.

### Example 3: Pharmacokinetics and Drug Metabolism in Rats

An in vitro study to evaluate pharmacokinetics with combination Compound 1 and fesoterodine was conducted.

The plasma concentration-time profile was evaluated for fesoterodine active metabolite desfesoterodine and Compound 1 in male Sprague Dawley rats following single-dose intravenous administration of fesoterodine (0.25 mg/kg) and Compound 1 (0.5 mg/kg and 1.5 mg/kg) alone and single-dose co-administration. Plasma exposures of desfesoterodine were similar following administration of fesoterodine alone and in combination with Compound 1. The PK parameters of Compound 1 were comparable at the 1.5 mg/kg dose single-agent administration and co-administration with fesoterodine (Table 3).

Group 1 (fesoterodine 0.25 mg/kg) active metabolite desfesoterodine concentrations were quantifiable from 0.033 hours to 6 hours.

Group 2 (Compound 1 1.5 mg/kg) Compound 1 pharmacokinetics showed moderate plasma clearance with approximately 50% of the normal liver blood flow in rats (55 mL/min/kg), moderate Vss of approximately 2.4-fold total body water (0.7 L/kg), and 0.83-hour terminal elimination plasma half-life.

Group 3 (Compound 1 0.5 mg/kg co-administered with fesoterodine 0.25 mg/kg) active metabolite desfesoterodine concentrations were quantifiable from 0.033 hours to 6 hours. Compound 1 showed moderate plasma clearance of approximately 40% of the normal liver blood flow in rats (55 mL/min/kg), moderate Vss of approximately 3-fold total body water (0.7 L/kg), and 2.18-hour terminal elimination plasma half-life.

Group 4 (Compound 1 1.5 mg/kg co-administered with fesoterodine 0.25 mg/kg) active metabolite desfesoterodine concentrations were quantifiable from 0.033 hours to 6 hours. Compound 1 showed moderate plasma clearance of approximately 42% of the normal liver blood flow in rats (55 mL/min/kg), moderate Vss of approximately 2.7-fold total body water (0.7 L/kg), and 1.24-hour terminal elimination plasma half-life.

TABLE 3
Compound 1 and Desfesoterodine Plasma Pharmacokinetic Parameters in Rats

| | | Dose | C₀ | AUCₗₐₛₜ | T_{1/2} | CL | V_{SS} |
|---|---|---|---|---|---|---|---|
| Analyte | Route | (mg/kg) | (ng/mL) | (h*ng/mL) | (h) | (mL/min/kg) | (L/kg) |
| DF | IV | Fesoterodine: 0.25 | 27.09 | 15.41 | — | — | — |
| | | Fesoterodine: 0.25 | 34.63 | 13.05 | — | — | — |
| | | Compound 1: 0.5 | | | | | |
| | | Fesoterodine: 0.25 | 38.71 | 13.73 | — | — | — |
| | | Compound 1: 1.5 | | | | | |
| C1 | IV | Compound 1: 1.5 | 1523.09 | 917.18 | 0.83 | 27.21 | 1.71 |
| | | Fesoterodine: 0.25 | 388.18 | 371.97 | 2.18 | 21.84 | 2.1 |
| | | Compound 1: 0.5 | | | | | |
| | | Fesoterodine: 0.25 | 1203.02 | 1068.93 | 1.24 | 23.35 | 1.86 |
| | | Compound 1: 1.5 | | | | | |

Abbreviations:
DF = desfesoterodine
AUCₗₐₛₜ = area under the curve,
C₀ = concentration at time “0” for initial IV bolus dose extrapolated from initial measured plasma concentrations,
CL = clearance,
h = hour,
IV = intravenous,
kg = kilogram,
L = liter,
mg = milligram,
mL = milliliter,
ng = nanogram,
V_{SS} = volume of distribution at steady-state

### Example 4: Compound 1 Effect on MPTP-Induced Dyskinesia

The neurotoxin 1-Methyl-4-phenyl-1,2,3,6-tetrahydropyridine (MPTP) was identified as a compound that selectively induced degeneration of dopaminergic neurons in the substantia nigra when it was discovered as a contaminant in heroin and users presented with clinical symptoms indistinguishable from PD. It is now used in non-human primates to model primary parkinsonian motor symptom phenotypes and subsequent response to therapies such as L-dopa including dyskinesia-thus it can be used as foundational model for LID. See Huat 2012. (Huot P, Johnston T H, Koprich J B, Fox S H, Brotchie J M. L-DOPA pharmacokinetics in the MPTP-lesioned macaque model of Parkinson's disease. Neuropharmacology. 2012 October; 63(5):829-36.) Having both face validity and construct validity, the MPTP non-human primate model is considered the animal model of PD motor symptoms with the highest translational relevance.

A study was conducted in seven female MPTP-lesioned cynomolgus macaques that received chronic repeat treatment with L-dopa and in response manifested stable and reproducible dyskinesia that was choreic and dystonic in nature. Macaque behaviors were assessed following a single 1 mg/kg dose of Compound 1 or vehicle in combination with high-dose of L-dopa delivered as Madopar™ with a dose titrated for LID symptoms in each test animal. The ‘on time’ therapeutic effects and abnormal LID movements were assessed for 6 hours following administration of L-dopa. A single Compound 1 oral dose of 1 mg/kg or vehicle was administered at the same time as L-dopa at the start of behavioral observations. All animals received all treatments in a cross-over design with a minimum of 72 hours between repeat treatments in each animal. The therapeutic and LID behaviors were scored by trained raters supervised by a neurologist in a blinded fashion using high-definition video recordings and the non-human primate dyskinesia rating scale. See Fox 2012. (Fox S H, Johnston T H, Li Q, Brotchie J, Bezard E. A critique of available scales and presentation of the Non-Human Primate Dyskinesia Rating Scale. Mov Disord. 2012 Sep. 15; 27(11):1373-8.)

Compound 1 (1 mg/kg, PO) provided significant anti-dyskinetic actions reducing median levels of dyskinesia during the 0-1 h period by 91% (median levels 11 cf. 1; vehicle cf. Compound 1, P<0.001). A robust reduction in LID was apparent in six of the seven animals included in final analyses. Cumulated over the 0-2 h period, median levels of dyskinesia were reduced by 42% such that levels were non-disabling (median levels 33 cf. 19; vehicle cf. Compound 1, P=0.0156). This sizeable decrease in peak-effect dyskinesia was further apparent as a beneficial decrease (by 41%) in the duration of ‘bad’ on-time (on-time associated with disabling dyskinesia) and a significant increase (by 143%) in the duration of ‘good’ on-time (on-time associated with a lack of, or only non-disabling dyskinesia). Compound 1 produced no reduction in the anti-parkinsonian benefit of L-DOPA or the overall duration of on-time associated with L-DOPA.

These results of the MPTP-induced dyskinesia macaque study provide strong evidence of the treatment potential of Compound 1 for LID in PD patients.

### Example 5: Pharmacokinetics of Compound 1 Following Oral Administration to Cynomolgus Monkeys

The following example characterized the pharmacokinetics (PK) of Compound 1 in Cynomolgus Monkey following single oral (PO) Compound 1 dose administration. The compound was administered via oral gavage at 0.3 mg/kg and 1 mg/kg, formulated as solution in saline. After dosing, plasma was collected to characterize the PK profiles of Compound 1. No adverse effects were observed during the study. The results are summarized in Table 4.

TABLE 4
Summary of Mean Plasma Exposure and
PK parameters of Compound 1 (n = 3)

| | Dose | T_{1/2} | Cₘₐₓ | Tₘₐₓ | AUC_{0-inf} | AUC₀₋ₜ |
|---|---|---|---|---|---|---|
| Group | (mg/kg) | (h) | (ng/mL) | (h) | (h*ng/ml) | (h*ng/mL) |
| A | 0.3 | 2.82 | 216 | 0.417 | 212 | 208 |
| B | 1 | 2.28 | 686 | 1.00 | 988 | 982 |

AUC₀₋ₜ, t = 24 h post dose for all the animals

### Example 6: Clinical Observations and Pharmacokinetics of Compound 1 and Fesoterodine

Compound 1 and fesoterodine fumarate, alone and in combination, were orally administered to male beagle dogs and clinical observations and plasma concentrations of Compound 1 and desfesoterodine were observed. The study was conducted over a 7 day period and the levels of Compound 1 and desfesoterodine (active fesoterodine metabolite) were measured over 24 hours on day 1 and day 7.
Group 1: Compound 1 (1.5 mg/kg/day PO)
Group 2: fesoterodine fumarate (3 mg/kg/day PO)
Group 3: Compound 1 (1.5 mg/kg/day PO) and fesoterodine fumarate (3 mg/kg/day PO)

Animals in Group 1, Group 2, and Group 3 were observed after dosing, as shown in Table 9. Group 1, which was treated only with Compound 1, showed peripheral side effects. However, when treated with both Compound 1 and fesoterodine (Group 3) no peripheral side effects were observed. Group 3 only showed miosis. These data show that the administration of fesoterodine fumarate substantially reduced the peripheral effects (Group 3) that were observed in subjects administered Compound 1 alone (Group 1).

TABLE 5
Individual and mean concentration of Compound 1 after treatment with Compound 1 (Group 1)
Individual and Mean Concentration of Compound 1
Plasma concentration (ng/ml)

| | | | | | | | | |
|---|---|---|---|---|---|---|---|---|
| Time (h) | D1001 | D1002 | D1003 | D1004 | D1005 | Mean | SD | CV (%) |

Compound 1 - Day 1

| | | | | | | | | | |
|---|---|---|---|---|---|---|---|---|---|
| 0.25 | BQL | BQL | BQL | BQL | BQL | ND | ± | ND | ND |
| 0.5 | BQL | BQL | BQL | BQL | BQL | ND | ± | ND | ND |
| 1 | 320 | 132 | 77.4 | 33.9 | 1328 | 378.26 | ± | 542.0205 | 143.2931 |
| 2 | 263 | 171 | 169 | 431 | 405 | 287.8 | ± | 125.1127 | 43.47212 |
| 4 | 60.3 | 87.8 | 65.7 | 202 | 77.4 | 98.64 | ± | 58.75145 | 59.56149 |
| 8 | BQL | 10.9 | BQL | 23 | 10.3 | 14.73333 | ± | 7.165426 | 48.63411 |
| 24 | BQL | BQL | BQL | BQL | BQL | ND | ± | ND | ND |

Compound 1 - Day 7 ±

| | | | | | | | | | |
|---|---|---|---|---|---|---|---|---|---|
| 0 | BQL | BQL | BQL | BQL | BQL | ND | ± | ND | ND |
| 0.25 | BQL | BQL | BQL | BQL | BQL | ND | ± | ND | ND |
| 0.5 | BQL | BQL | BQL | BQL | BQL | ND | ± | ND | ND |
| 1 | 370 | 586 | 263 | 318 | 398 | 387 | ± | 122.6254 | 31.68616 |
| 2 | 271 | 584 | 667 | 386 | 445 | 470.6 | ± | 157.389 | 33.44433 |
| 4 | 62.9 | 140 | 139 | 102 | 53.5 | 99.48 | ± | 40.81148 | 41.02481 |
| 8 | 9.81 | 19 | 25.6 | 21.4 | 9.68 | 17.098 | ± | 7.116012 | 41.61897 |
| 24 | BQL | BQL | BQL | BQL | BQL | ND | ± | ND | ND |

TABLE 6
Individual and mean concentration of Compound 1 after treatment
with Compound 1 and fesoterodine fumarate (Group 3)
Individual and Mean Concentration of Compound 1
Plasma concentration (ng/ml)

| | | | | | | | | |
|---|---|---|---|---|---|---|---|---|
| Time (h) | D3001 | D3002 | D3003 | D3004 | D3005 | Mean | SD | CV (%) |

Compound 1 + fesoterodine fumarate - Day 1

| | | | | | | | | | |
|---|---|---|---|---|---|---|---|---|---|
| 0.00 | BQL | BQL | BQL | BQL | BQL | ND | ± | ND | ND |
| 0.250 | BQL | BQL | BQL | BQL | BQL | ND | ± | ND | ND |
| 0.500 | BQL | BQL | BQL | BQL | BQL | ND | ± | ND | ND |
| 0.750 | 832 | 31.5 | 880 | 127 | 296 | 433 | ± | 398 | 91.8 |
| 1.00 | 716 | 38.5 | 794 | 175 | 557 | 456 | ± | 334 | 73.2 |
| 2.00 | 254 | 113 | 356 | 1290 | 2571 | 917 | ± | 1034 | 113 |
| 4.00 | 41.7 | 17.9 | 58.2 | 791 | 1088 | 399 | ± | 504 | 126 |
| 8.00 | 17.3 | 380 | 121 | 124 | 169 | 162 | ± | 134 | 82.5 |
| 24.0 | BQL | 4.56 | BQL | BQL | BQL | ND | ± | ND | ND |

Compound 1 + fesoterodine fumarate - Day 7

| | | | | | | | | | |
|---|---|---|---|---|---|---|---|---|---|
| 0.00 | BQL | BQL | BQL | BQL | 6.28 | ND | ± | ND | ND |
| 0.250 | BQL | BQL | BQL | BQL | BQL | ND | ± | ND | ND |
| 0.500 | BQL | BQL | BQL | BQL | BQL | ND | ± | ND | ND |
| 0.750 | 49.8 | 19.6 | 469 | 304 | 376 | 244 | ± | 200 | 82.0 |
| 1.00 | 151 | 30.3 | 759 | 508 | 662 | 422 | ± | 318 | 75.4 |
| 2.00 | 468 | 54.2 | 982 | 1109 | 1645 | 852 | ± | 612 | 71.8 |
| 4.00 | 1177 | 67.1 | 719 | 827 | 873 | 733 | ± | 409 | 55.8 |
| 8.00 | 183 | 145 | 114 | 111 | 124 | 135 | ± | 29.8 | 22.0 |
| 24.0 | BQL | BQL | BQL | BQL | BQL | ND | ± | ND | ND |

TABLE 7
Individual and mean concentration of desfesoterodine after treatment
with Compound 1 and fesoterodine fumarate (Group 3)
Individual and Mean Concentration of desfesoterodine
Plasma concentration (ng/ml)

| | | | | | | | | |
|---|---|---|---|---|---|---|---|---|
| Time (h) | D3001 | D3002 | D3003 | D3004 | D3005 | Mean | SD | CV (%) |

Compound 1 + fesoterodine fumarate - Day 1

| | | | | | | | | | |
|---|---|---|---|---|---|---|---|---|---|
| 0.00 | BQL | BQL | BQL | BQL | BQL | ND | ± | ND | ND |
| 0.250 | 225 | 35.2 | 333 | 23.3 | 9.33 | 125 | ± | 146 | 116 |
| 0.500 | 154 | 58.0 | 257 | 35.9 | 52.4 | 111 | ± | 93.6 | 84.0 |
| 0.750 | 136 | 35.1 | 203 | 64.7 | 61.3 | 100 | ± | 68.7 | 68.7 |
| 1.00 | 96.0 | 30.6 | 149 | 15.8 | 41.5 | 66.6 | ± | 55.1 | 82.8 |
| 2.00 | 32.4 | 17.2 | 74.2 | 10.7 | 45.5 | 36.0 | ± | 25.3 | 70.2 |
| 4.00 | 7.08 | 3.69 | 17.0 | 5.38 | 13.3 | 9.29 | ± | 5.64 | 60.7 |
| 8.00 | 1.41 | 4.22 | 3.82 | BQL | 1.67 | 2.78 | ± | 1.45 | 52.0 |
| 24.0 | BQL | BQL | BQL | BQL | BQL | ND | ± | ND | ND |

Compound 1+ fesoterodine fumarate - Day 7

| | | | | | | | | | |
|---|---|---|---|---|---|---|---|---|---|
| 0.00 | BQL | BQL | BQL | BQL | BQL | ND | ± | ND | ND |
| 0.250 | 29.1 | 60.6 | 407 | 149 | 270 | 183 | ± | 156 | 85.3 |
| 0.500 | 103 | 77.7 | 318 | 197 | 311 | 201 | ± | 112 | 55.9 |
| 0.750 | 84.3 | 66.4 | 294 | 102 | 191 | 148 | ± | 94.9 | 64.3 |
| 1.00 | 73.5 | 64.7 | 224 | 105 | 175 | 128 | ± | 68.8 | 53.6 |
| 2.00 | 45.8 | 42.6 | 149 | 63.2 | 102 | 80.5 | ± | 45.0 | 55.9 |
| 4.00 | 52.2 | 15.2 | 61.6 | 31.6 | 40.5 | 40.2 | ± | 18.0 | 44.8 |
| 8.00 | 10.6 | 3.92 | 8.16 | 2.93 | 4.06 | 5.93 | ± | 3.29 | 55.5 |
| 24.0 | BQL | BQL | BQL | BQL | BQL | ND | ± | ND | ND |

TABLE 8
Individual and mean concentration of desfesoterodine after treatment with fesoterodine fumarate (Group 2)
Individual and Mean Concentration of desfesoterodine
Plasma concentration (ng/ml)

| | | | | | | | | |
|---|---|---|---|---|---|---|---|---|
| Time (h) | D2001 | D2002 | D2003 | D2004 | D2005 | Mean | SD | CV (%) |

fesoterodine fumarate - Day 1

| | | | | | | | | | |
|---|---|---|---|---|---|---|---|---|---|
| 0.25 | 34.2 | 267 | 14 | BQL | 35.9 | 87.775 | ± | 119.8967 | 136.5955 |
| 0.5 | 68.4 | 176 | 75.3 | 23.2 | 33.7 | 75.32 | ± | 60.48361 | 80.30219 |
| 1 | 31.9 | 104 | 49.5 | 67.6 | 13.9 | 53.38 | ± | 34.63996 | 64.89315 |
| 2 | 15.6 | 48.4 | 15.7 | 41.4 | 5.38 | 25.296 | ± | 18.5464 | 73.31753 |
| 4 | 12.9 | 15.4 | 3.93 | 14.9 | 3.35 | 10.096 | ± | 5.97079 | 59.14015 |
| 8 | 4.42 | 1.74 | 2.96 | 4.15 | BQL | 3.3175 | ± | 1.228125 | 37.0196 |
| 24 | BQL | BQL | BQL | BQL | BQL | ND | ± | ND | ND |

fesoterodine fumarate - Day 7

| | | | | | | | | | |
|---|---|---|---|---|---|---|---|---|---|
| 0 | 1.13 | BQL | BQL | BQL | BQL | ND | ± | ND | ND |
| 0.25 | 15.3 | 315 | 121 | 47.3 | 18.4 | 103.4 | ± | 125.7161 | 121.5823 |
| 0.5 | 318 | 274 | 154 | 75.6 | 16.6 | 167.64 | ± | 127.8584 | 76.26962 |
| 1 | 173 | 232 | 111 | 65.7 | 11.8 | 118.7 | ± | 86.71978 | 73.05794 |
| 2 | 91.9 | 145 | 72.6 | 57.8 | 25.8 | 78.62 | ± | 44.27846 | 56.31959 |
| 4 | 51.4 | 48.6 | 44 | 40.8 | 43.5 | 45.66 | ± | 4.26122 | 9.332502 |
| 8 | 8.12 | 7.08 | 6.52 | 13.8 | 4.81 | 8.066 | ± | 3.422189 | 42.42734 |
| 24 | BQL | BQL | BQL | BQL | BQL | ND | ± | ND | ND |

TABLE 9
Clinical observations after treatment of Group 1, Group 2, and Group 3

| | Animal | |
|---|---|---|
| Group | ID | Clinical Observation |
| 1 | D1001 | For D1001, soft feces were found 1 hr 45 min after dosing on day 1. |
| | | For D1001, 10 mL chyme was found 55 min after dosing on day 3. |
| | | For D1001, 30 mL liquid feces and ptyalism were found 80 min after |
| | | dosing on day 3. |
| | | For D1001, ptyalism was found 2 hr after dosing on day 3. 8 hr recover |
| | | For D1001, 20 mL chyme and ptyalism were found 3.5 hr after dosing |
| | | on day 3. |
| | | For D1001, 15 mL chyme was found 2 hr after dosing on day 4. |
| | | For D1001, ptyalism and 15 mL liquid feces were found 3 hr after |
| | | dosing on day 4. |
| | | For D1001, 15 mL chyme and soft feces were found 2 hr after dosing on |
| | | day 5. |
| | | For D1001, ptyalism was found 30 min after dosing on day 6. 8 hr |
| | | recover. |
| | | For D1001, ptyalism and soft feces were found 1 hr after dosing on day 6. |
| | | For D1001, 15 mL chyme, 10 mL liquid feces, tremor and ptyalism were |
| | | found 2 hr after dosing on day 6. |
| | | For D1001, 5 mL chyme and ptyalism were found 3 hr after dosing on |
| | | day 6. |
| | | For D1001, ptyalism was found 1 hr after dosing on day 7. 4 hr recover. |
| | | For D1001, miosis was found 1 hr after dosing on day 7. 8 hr recover. |
| | | For D1001, about 5 mL liquid feces and about 3 mL yellow liquid |
| | | vomitus were found 2 hr after dosing on day 7. |
| | D1002 | For D1002, 10 mL chyme was found 55 min after dosing on day 3. |
| | | For D1002, 10 mL liquid feces and ptyalism were found 80 min after |
| | | dosing on day 3. |
| | | For D1002, 10 mL chyme and 5 mL liquid feces were found 90 min |
| | | after dosing on day 3. |
| | | For D1002, ptyalism was found 2 hr after dosing on day 3. 8 hr recover |
| | | For D1002, 5 mL chyme, 5 mL liquid feces and ptyalism were found 3.5 |
| | | min after dosing on day 3. |
| | | For D1002, 15 mL chyme and 10 mL liquid feces were found 2 hr after |
| | | dosing on day 4. |
| | | For D1002, ptyalism and 5 mL liquid feces were found 3 hr after dosing |
| | | on day 4. |
| | | For D1002, 20 mL chyme and 10 mL liquid feces were found 2 hr after |
| | | dosing on day 5. |
| | | For D1002, ptyalism was found 30 min after dosing on day 6. 8 hr |
| | | recover. |
| | | For D1002, 10mL liquid feces and ptyalism were found 1 hr after dosing |
| | | on day 6. |
| | | For D1002, 20 mL chyme, 10 mL liquid feces and ptyalism were found |
| | | 2 hr after dosing on day 6. |
| | | For D1002, ptyalism was found 1 hr after dosing on day 7. 4 hr recover. |
| | | For D1002, about 1 mL liquid feces was found 2 hr after dosing on |
| | | day 7. |
| | D1003 | For D1003, 5 mL liquid feces and ptyalism were found 80 min after |
| | | dosing on day 3. |
| | | For D1003, ptyalism was found 2 hr after dosing on day 3. 8 hr recover |
| | | For D1003, 10 mL chyme, 5 mL liquid feces and ptyalism were found |
| | | 3.5 hr after dosing on day 3. |
| | | For D1003, 10 mL liquid feces and ptyalism were found 4 hr after |
| | | dosing on day 3. |
| | | For D1003, 20 mL liquid feces and ptyalism were found 4.5 hr after |
| | | dosing on day 3. |
| | | For D1003, 10 mL chyme and ptyalism were found 2 hr after dosing on |
| | | day 4. |
| | | For D1003, soft feces was found 3 hr after dosing on day 4. |
| | | For D1003, 5 mL chyme was found 2 hr after dosing on day 5. |
| | | For D1003, ptyalism was found 30 min after dosing on day 6. 8 hr |
| | | recover. |
| | | For D1003, 15mL chyme, 30mL liquid feces, ptyalism and tremor were |
| | | found 1 hr after dosing on day 6. |
| | | For D1003, 30 mL liquid feces, ptyalism and tremor were found 2 hr |
| | | after dosing on day 6. |
| | | For D1003, 10 mL chyme was found 3 hr after dosing on day 6. |
| | | For D1003, ptyalism was found 1 hr after dosing on day 7. 4hr recover. |
| | | For D1003, about 10 mL liquid feces was found 2 hr after dosing on day 7. |
| | D1004 | For D1004, 15 mL chyme and soft feces were found 55 min after dosing |
| | | on day 3. |
| | | For D1004, 2 mL liquid feces and ptyalism were found 80 min after |
| | | dosing on day 3. |
| | | For D1004, 20 mL chyme was found 90 min after dosing on day 3. |
| | | For D1004, ptyalism was found 2 hr after dosing on day 3. 8 hr recover |
| | | For D1004, ptyalism was found 2 hr after dosing on day 4. |
| | | For D1004, 8 mL liquid feces was found 3 hr after dosing on day 4. |
| | | For D1004, soft feces was found 220 min after dosing on day 4. |
| | | For D1004, 15 mL chyme and 20 mL liquid feces were found 2 hr after |
| | | dosing on day 5. |
| | | For D1004, ptyalism was found 30 min after dosing on day 6. 8hr |
| | | recover. |
| | | For D1004, 10 mL liquid feces and ptyalism were found 1 hr after |
| | | dosing on day 6. |
| | | For D1004, 20 mL chyme and ptyalism were found 2 hr after dosing on |
| | | day 6. |
| | | For D1004, 5 mL chyme was found 3 hr after dosing on day 6. |
| | | For D1004, ptyalism was found 1 hr after dosing on day 7. 4 hr recover. |
| | | For D1004, about 3 mL yellow liquid vomitus was found 1 hr after |
| | | dosing on day 7. |
| | D1005 | For D1005, about 4 mL white foamy mucoid vomitus was found 40 min |
| | | after dosing on day 1. |
| | | For D1005, soft feces was found 1 hr after dosing on day 1. |
| | | For D1005, about 8 mL yellow foamy mucoid vomitus was found 1 hr |
| | | 10 min after dosing on day 1. |
| | | For D1005, ptyalism was found 1 hr 20 min after dosing on day 1. |
| | | For D1005, 20 mL chyme was found 45 min after dosing on day 3. |
| | | For D1005, soft feces was found 80 min after dosing on day 3. |
| | | For D1005, 20 mL liquid feces was found 3.5 hr after dosing on day 3. |
| | | For D1005, 15 mL chyme was found 40 min after dosing on day 4. |
| | | For D1005, 10 mL chyme and 10 mL liquid feces were found 2 hr after |
| | | dosing on day 4. |
| | | For D1005, 8 mL chyme and ptyalism were found 3 hr after dosing on |
| | | day 4. |
| | | For D1005, 15 mL chyme and soft feces were found 2 hr after dosing on |
| | | day 5. |
| | | For D1005, 15 mL chyme, 30 mL liquid feces and ptyalism were found |
| | | 1 hr after dosing on day 6. |
| | | For D1005, 15 mL chyme was found 2 hr after dosing on day 6. |
| | | For D1005, about 8 mL liquid feces was found 2 hr after dosing on day 7. |
| 2 | D2001 | For D2001, miosis was found 1 hr 10 min after dosing on day 1. 2 hr |
| | | 30min recover. |
| | D2002 | For D2002, miosis was found 1 hr 10 min after dosing on day 1. 2 hr |
| | | 30 min recover. |
| | D2003 | none |
| | D2004 | none |
| | D2005 | none |
| 3 | D3001 | none |
| | D3002 | none |
| | D3003 | none |
| | D3004 | none |
| | D3005 | none |

### Example 7: Oral Administration of Compound 1 (Single Ascending Dose) With or Without Administration of Fesoterodine Fumarate in Healthy Subjects

Compound 1 was administered in a single ascending dose (SAD) study conducted in approximately 54 healthy adult volunteers to investigate Compound 1 pharmacokinetics, safety, and tolerability. The study was conducted in two groups.

Group 1 evaluated multiple dose cohorts administered Compound 1 in a single ascending dose (SAD) manner to establish a maximum tolerated dose (MTD) using the doses described in Table 10.

TABLE 10
Compound doses levels in Group 1

| | Compound 1 |
|---|---|
| Cohort | Dose |
| 1 | 1 mg |
| 2 | 3 mg |
| 3 | 10 mg |
| 4 | 20 mg |
| 5 | 40 mg |
| 6 | 60 mg |

MTD was established at the dose which produced mild to moderate cholinergic adverse effects. Cholinergic effects were monitored for each dose, including pupillometry and salivation.

Group 2 evaluated tolerability of Compound 1 at Group 1 MTD with or without fesoterodine fumarate co-administration. Subjects received 8 mg of fesoterodine fumarate tablet or matched placebo tablet 4 hours prior to administration of 40 mg of Compound 1.

Results: Safety—In Group 1, a total of 95 treatment-emergent adverse events (TEAEs) were observed by 22 (61.6%) of the 36 subjects who received any dose of compound 1. About 50.0%, 33.3%, 50.0%, 50.0%, 50.0%, 83.3%, and 100% subjects in 0 mg (placebo), 1 mg, 3 mg, 10 mg, 20 mg, 40 mg, and 60 mg treatment groups, respectively, reported TEAEs. There were no deaths or serious TEAEs reported, and no subjects discontinued the study due to a TEAE. In Group 2, a total of 9 TEAEs were reported by 100.0% subjects who received Compound 1+placebo and 20 TEAEs were reported by 83.3% of 6 subjects who received Compound 1+fesoterodine fumarate. There were no deaths, serious or severe TEAEs reported. No subject was discontinued due to a TEAE. The most frequent TEAEs observed in the placebo treatment group were salivary hypersecretion and dizziness. In the fesoterodine treatment group, dizziness and presyncope were observed.

Similar to what was observed in actively treated subjects in Group 1 cohorts, all the TEAEs were experienced on Day 1 (i.e., the day of dosing) in the Compound 1+placebo treatment group in Group 2. Conversely, in the Compound 1 +fesoterodine fumarate treatment group, the TEAEs in subjects had onset at least one day after dosing (Days 2 to 5). The delay of onset of these TEAEs in this treatment group is due to administration of extended-release formulation of fesoterodine fumarate prior to Compound 1 (i.e., consistent with muscarinic blockade).

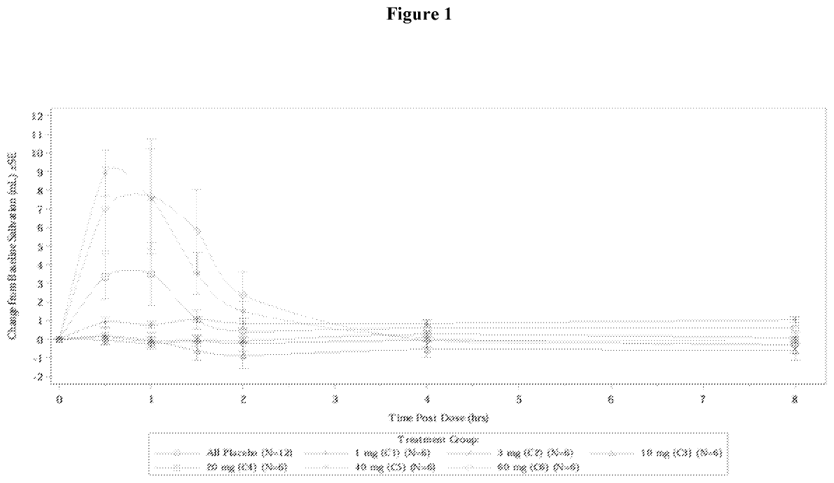

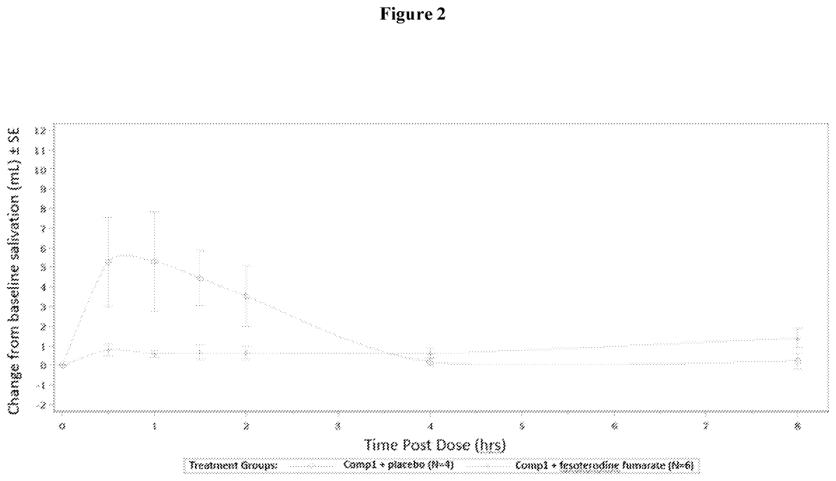

Pharmacodynamics—In Group 1, increase in salivation was observed with increasing dose of Compound 1, as shown in FIG. 1. In Group 2, the increase in salivation observed post Compound 1 dosing was not observed in the subjects pre-dosed with fesoterodine fumarate compared with those pre-treated with placebo (FIG. 2), indicating a successful blockade by fesoterodine of peripheral muscarinic effects of Compound 1.

Pharmacokinetics—In Group 1, the compound 1 concentration in CSF at 1hour post-dose increased with increasing dose and was 19.15 ng/ml (Cohort 3—10 mg, N=1), 32.27 ng/ml mean (Cohort 4—20 mg, N=2), 50.30 ng/ml mean (Cohort 5—40 mg, N=2) and 59.15 ng/ml (Cohort 6—60 mg, N=1). In Group 2, the mean compound 1 concentration in CSF at 1 hour post-dose was 43.29 ng/ml and when dosed after fesoterodine fumarate, was 56.96 ng/mL. The mean desfesoterodine concentration in CSF at 1hour after administration of compound 1 was 121.44 pg/mL, and the mean ratio of CSF Compound 1 to desfesoterodine was 469:1.

Conclusions: The administration of Compound 1 following single oral doses ranging from 1 mg to 40 mg was generally safe and well tolerated in healthy subjects, with 40 mg established as the MTD of Compound 1 when administered alone. Objective increase in salivation was observed with increasing dose of Compound 1. Overall, in Group 2, pre-treatment with extended-release fesoterodine resulted in a delay in TEAE onset following Compound 1 dosing. Furthermore, pre-treatment with fesoterodine nearly abolished both subjective reports of hypersalivation and the objective increase in saliva volume observed with Compound 1, consistent with fesoterodine having blocked the peripheral muscarinic effect of Compound 1. In this study, the absorption of Compound 1 was generally fast with median tmax observed between 0.65 to 1.17-hours. Following co-administration of fesoterodine fumarate, the exposure of Compound 1 was higher by 30.0%, 29.0%, and 50.0% for AUC0-t, AUC0-inf, and Cmax, respectively. However, only the increase in Cmax was considered statistically significant (p-value: 0.0347).

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A method of treating a dyskinesia in a patient in need thereof, the method comprising administering:
(a) a therapeutically effective amount of Compound 1, or a pharmaceutically acceptable salt thereof, and
(b) a therapeutically effective amount of fesoterodine, or a pharmaceutically acceptable salt thereof.

2. The method of claim 1, wherein the dyskinesia is levodopa-induced dyskinesia.

3. The method of any one of claims 1-2, wherein the patient is diagnosed with Parkinson's disease.

4. The method of any one of claims 1-3, wherein about 5 mg to about 800 mg of Compound 1, or a pharmaceutically acceptable salt thereof is administered to the patient.

5. The method of claim 4, wherein about 20 mg to about 80 mg of Compound 1, or a pharmaceutically acceptable salt thereof is administered to the patient.

6. The method of claim 4, wherein about 60 mg of Compound 1, or a pharmaceutically acceptable salt thereof is administered to the patient.

7. The method of any one of claims 1-6, wherein the administration provides a therapeutically effective steady-state plasma concentration of Compound 1.

8. The method of claim 7, wherein the therapeutically effective steady-state plasma concentration of Compound 1 is about 100 ng/ml to about 2500 ng/mL.

9. The method of claim 7, wherein the therapeutically effective steady-state plasma concentration of Compound 1 is about 600 ng/ml.

10. The method of any one of claims 1-9, wherein about 1 mg to about 50 mg of fesoterodine, or a pharmaceutically acceptable salt thereof is administered to the patient.

11. The method of claim 10, wherein about 8 mg of fesoterodine, or a pharmaceutically acceptable salt thereof is administered.

12. The method of claim 10, wherein about 24 mg of fesoterodine, or a pharmaceutically acceptable salt thereof is administered.

13. The method of any one of claims 1-12, wherein the administration provides a therapeutically effective steady-state plasma concentration of desfesoterodine (i.e., an amount sufficient to reduce peripheral side effects associated with administration of Compound 1).

14. The method of claim 13, wherein the therapeutically effective steady-state plasma concentration of desfesoterodine is about 5 ng/mL to about 30 ng/mL.

15. The method of any one of claims 1-14, wherein the Compound 1, or a pharmaceutically acceptable salt thereof is administered once per day.

16. The method of any one of claims 1-14, wherein the Compound 1, or a pharmaceutically acceptable salt thereof is administered twice per day.

17. The method of any one of claims 1-14, wherein the Compound 1, or a pharmaceutically acceptable salt thereof is administered three times per day.

18. The method of any one of claims 1-17, wherein the fesoterodine, or a pharmaceutically acceptable salt thereof is administered once per day.

19. The method of any one of claims 1-17, wherein the fesoterodine, or a pharmaceutically acceptable salt thereof is administered twice per day.

20. The method of any one of claims 1-17, wherein the fesoterodine, or a pharmaceutically acceptable salt thereof is administered three times per day.

21. The method of any one of claims 1-20, wherein the Compound 1 and fesoterodine are administered in separate pharmaceutical compositions.

22. The method of any one of claims 1-21, wherein the Compound 1 and fesoterodine are orally administered.

23. The method of any one of claims 1-22, wherein the fesoterodine, or a pharmaceutically acceptable salt thereof is administered in an extended release composition.

24. The method of any one of claims 1-23, wherein the patient is administered the Compound 1 at least one hour after the patient is administered the fesoterodine.

25. The method of claim 24, wherein the patient is administered the Compound 1 about four hours after the patient is administered the fesoterodine.

26. The method of any one of claims 1-25, wherein the administration provides a Compound 1 to desfesoterodine plasma concentration ratio of about 10:1 to about 1000:1.

27. The method of any one of claims 1-25, wherein the administration provides a Compound 1 to desfesoterodine plasma concentration ratio of about 100:1.

28. The method of any one of claim 1-23 or 26-27, wherein the Compound 1 and fesoterodine are administered in the same pharmaceutical composition.

29. The method of any one of claims 1-28, wherein the Compound 1 comprises the hydrochloride salt of formula:

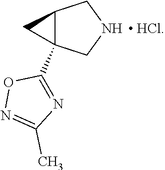

30. The method of any one of claims 1-29, wherein the fesoterodine, or a pharmaceutically acceptable salt thereof comprises fesoterodine fumarate.

31. The method of any one of claims 1-30, wherein the dose and administration schedule of fesoterodine are selected to reduce the peripheral side effects of administering Compound 1 to the patient.
